(19) United States
(12) Patent Application Publication
(10) Pub. No.: US 2015/0351685 A1
(43) Pub. Date: Dec. 10, 2015

(54) PERSONAL WEIGHT MANAGEMENT METHOD

(71) Applicant: Thomas Norbert Weichselbaumer, Steyr (AT)

(72) Inventor: Thomas Norbert Weichselbaumer, Steyr (AT)

(21) Appl. No.: 14/438,523

(22) PCT Filed: Oct. 23, 2013
(86) PCT No.: PCT/EP2013/072214

§ 371 Date: Aug. 17, 2015

(30) Foreign Application Priority Data
Oct. 24, 2012 (EP) 12189844.9

(51) Int. Cl. A61B 5/00; G09B 19/00; A61B 5/11; G06F 19/00

(52) U.S. Cl. CPC A61B 5/4866; G06F 19/3475; G09B 19/0092; A61B 5/1118

(57) ABSTRACT

An electronic device and method for personal weight management are disclosed, in which a time measuring device is used to measure abstinence periods between consecutive times that a person consumes food and/or beverages containing chemical energy and these intervals are incorporated into the weight management. In order to enable a method that is as simple as possible, but is nevertheless effective and individually tailored to the person, it is proposed that a time value, which depends on the lipid metabolism—in particular on the lipolysis—that is present during the abstinence period, be subtracted from the respective measured abstinence periods and that the results calculated from this be incorporated into the weight management.

### FIELD OF THE INVENTION

The invention relates to a device and a method for personal weight management in which a time-measuring device is used to measure abstinence periods between the successive times a person consumes food and/or beverages containing chemical energy and these times are incorporated into the weight management.

### BACKGROUND OF THE INVENTION

Numerous methods for personal weight management are known from the prior art (for example including measures for losing, gaining, and/or stabilizing weight), in which various time intervals are measured and taken into account. In the case of a desired weight loss, these methods involve trying, among other things, to achieve the longest possible time intervals between consuming foods or beverages containing chemical energy—i.e. the longest possible abstinence periods—and to avoid consuming excess calories. To achieve this goal, among other things by means of an extremely wide variety of diet forms but also through increased physical activity, care is taken to achieve a negative calorie balance—on the assumption that this will induce the human body to cover the energy deficit by drawing on its fat tissue. Based on these principles, various approaches to weight loss are offered, which can in fact be used to achieve success with regard to a reduction of body weight—although only rarely does this turn out to be in the desired form over the long term. Specifically, the weight loss is thus often largely based on a decrease in muscle mass or is accompanied by such a decrease, which cannot be viewed as advantageous. Furthermore, current methods constitute a not insignificant psychological strain for the person involved, which is why the person often prematurely discontinues the methods of the prior art.

### SUMMARY OF THE INVENTION

The object of the invention, therefore, is to provide, based on the prior art, a method for personal weight management, which takes into account the metabolic processes of the body in an improved way in order to increase the effectiveness of the method and primarily also to ensure long-term success.

The invention attains the stated object in that a time value, which depends on the lipid metabolism—in particular the lipolysis—that is present during the abstinence period, is respectively subtracted from the measured abstinence periods and the results calculated from this are incorporated into the weight management.

If a time-measuring device is used to measure the abstinence periods between the successive times that a person consumes food and/or beverages containing chemical energy and these times are incorporated into the weight management, then it is possible to first investigate and also evaluate the dietary behavior of a person, among other things. During the abstinence period, the body thus does not consume any calories in the form of foods or beverages—the abstinence period ends with each calorie intake in the form of foods or beverages. Based on this information and the corresponding conclusions, it is possible to analyze certain metabolic processes in a person's body in relation to the weight management and depending on the desired effect, to use them in a targeted fashion, for example in order to reduce body fat.

The invention proposes using a time value that is subtracted from the measured abstinence periods. The results calculated from this are incorporated into the weight management. These time values depend on the lipid metabolism—in particular lipolysis—that is present during the abstinence period. The time value can thus be defined as the duration of time that passes from the beginning of an abstinence period until a person's metabolism has metabolically switched to primarily fat-burning mode. With regard to the insulin level, the time value can thus be seen as the time during which at the beginning of an abstinence period, the lipolysis in the cells of the fatty tissue is still inhibited by the postprandial insulin action as a result of a satisfying meal that contains chemical energy. If such a time value is subtracted from an abstinence period, then—naturally only if the abstinence period is greater than the time value—it is possible to first obtain a remaining duration of the abstinence period during which a fat-metabolizing metabolic state predominates. Advantageously, the results calculated from this can be taken into account in a weight management program.

The method according to the invention and its advantages will be explained in greater detail below in the particular example of lipid metabolism, i.e. the buildup and breakdown of fatty tissue in a person's body.

As is known, the regulation of the fat-, i.e. lipid, metabolism and therefore subsequent lipolysis occurs through the interplay of complex mechanisms, e.g. on the hormonal and enzymatic levels. Essentially, however, insulin can be said to play a central role in this. Thus for the sake of simplicity, explanations below should be provided particularly in relation to this hormone—the method, however, should not be understood as depending or being based solely on insulin. In general, it can be assumed that high insulin levels have an inhibiting effect on the lipolysis process, i.e. the breakdown of lipids in the fat cells (adipocytes) and thus the reduction of body fat, and promote the buildup of fatty tissue, whereas comparatively low insulin levels in the blood promote lipolysis. Since the insulin level rises after a carbohydrate-rich meal, a certain span of time must pass before it has fallen to such a level that a breakdown of lipids in the fat cells of the fatty tissue can predominate over the buildup of lipids in the fat cells. In the case of a desired reduction of body fat, it is therefore advantageous to know the time of this changeover in order to be able to use it in a targeted fashion.

Another significant advantage according to the invention is that in the method, it is possible to specify a dependence of the time value on the physical activity and on the sleep or wake state of the person during the respective abstinence period from which this time value is subtracted. In general, one can say that the time value in the sleep state is greater than in the wake state since the energy consumption of the body in the wake state—for example during the day at work—is at a higher level and because of the resulting, more quickly falling blood sugar level, the insulin level also falls more quickly. Physical activity also has a corresponding effect, which is why the time value, which is subtracted from the abstinence period with comparatively higher physical activity—for example in comparison to seated office work—, is likewise reduced in order to take into account a predominantly body fat-burning metabolic state that begins earlier.

In connection with physical activity, the method according to the invention can make advantageous use of numerous aspects—and can also take them into account in the weight management in a significantly targeted fashion. On the one hand, this is achieved by means of the effect that promotes the breakdown of body fat immediately during physical activity. On the other hand, this is also achieved by means of effect that continues to promote the breakdown of body fat immediately after physical activity, which effect continues until after the physical activity, the body once again ingests calories in the form of foods or beverages. It is therefore also possible with the method to detect the time interval between the end of an athletic activity and the time of the first subsequent calorie intake and to take this time interval into account in the weight management in a targeted fashion. In this case, the physical activity can also be advantageously taken into account with comparative independence from its type, intensity, etc. It is thus possible to detect and control individual differences with regard to the transition into different metabolic states. This therefore takes place in a way that is significantly improved in comparison to the prior art, which primarily uses the effect of a fat burning during athletic activity under precise conditions with regard to duration, intensity, etc.—but without taking into account the influence of athletic activity in the hours that follow and using it for the method in a targeted fashion.

It is also possible in turn—primarily by using an end of an abstinence period—to take into account when physical or athletic activity has little or no direct influence on a body fat-burning metabolic state—for example because the person has in the meantime consumed a beverage that contains carbohydrates. In this case, a predominantly fat-burning metabolic state can specifically only be achieved to a limited degree and/or only with significantly prolonged duration of exercise. It is known that increased physical activity, by increasing the muscle mass etc., can also increase the basal metabolic rate and thus facilitate a breakdown of body fat. The method can thus excel through its improved accounting for the metabolism and particularly also the lipolysis metabolism—whereas known methods often attempt to carry out a weight management based only on calorie counts. In addition, the method can advantageously encourage and/or motivate a person to increase his or her physical activity throughout the day, for example if a burning of fat is desired. This can significantly improve the sustainability of the weight management.

In the method according to the invention, it has surprisingly turned out that these complex interrelationships in the body's metabolism can be used in a comparatively simple, yet precise way. It is indeed necessary to take into account the fact that the metabolism varies individually in accordance with the type of physical activity, the gender, and the person, but individual deviations are comparatively slight. It is nevertheless possible according to the invention to individually define/establish the method and particularly time values by determining the gender, various physical states, and/or the physical fitness, dietary habits, muscle mass, etc. of a person; to regularly adapt to changes in this regard; and to incorporate these changes into the method. Advantageously, however, the method can also he used to avoid longer-lasting abstinence periods that result, for example, in undesirably high levels of musculature-reducing processes in the body.

A significant advantage of this weight management method is also that it can be carried out largely independent of the life rhythm or even dietary behavior and eating habits of a person. Consequently, it is practically immaterial what time of day meal times, sleep phases, or wake phases occur since the respective metabolic state of a person's body can be taken into account using a corresponding time value. Also, the different dietary habits of people due to their country, nationality, etc. play only a minor role since the method can detect particular metabolic functions and their effects independently of the composition and type of nutrition. The method is therefore able to excel due to a wide range of usability. The method can also be used to improve the individual responsibility of a person for his own weight management since according to the invention, the dependence of the body's metabolism on time values is taken into account in a simple way through the definition of abstinence periods. It is thus possible to achieve long-term success.

In accordance with the above, it is thus possible for the method according to the invention to also include the fact that the time value for the person is determined independently of their respective current calorie count in the course of a day. Consequently, with regard to the magnitude of the time value, it doesn't matter how many calories are consumed—primarily during the meal whose end marks the beginning of an abstinence period. In general, it should be emphasized that the relationship of calorie intake and calorie burning naturally plays a decisive role in avoiding overeating—but does not matter when determining the time value. This makes it easier to carry out the method—and is also a decisive difference from the prior art, in which all that happens is that a difference between the calories consumed and calories burned is calculated and an attempt is made to establish a weight management program based on these values—but without providing any information about particular metabolic processes, for example about the metabolic state of lipolysis, which in fact determines the actual reduction in body fat.

In general, it should be noted that the scope of the invention naturally also includes creating a weight management method for persons who wish to increase their body weight. To accomplish this, care is taken to make sure that their metabolism switches into a catabolic protein-burning or fat-burning state as little as possible. In this case, in the weight management, care is taken that a person consumes meals or energy-containing beverages on a regular basis and that the duration of any nutritional abstinence does not exceed the time value, which depends on the physical activity and the sleep or wake state of the person. The method can thus excel due to its ability to be used in a versatile way.

If the end of meals is taken as the beginning of an abstinence period only if in total, the meals in a day essentially cover the daily calorie requirements corresponding to the life circumstances of the person, then it is possible to make sure to avoid possibly disadvantageous metabolic processes in the course of a weight management program. It should be noted that in connection with covering the calorie requirements, it does not matter how many meals a person eats in the course of a day—this makes it possible to better take into account individual dietary behavior.

It is thus possible to avoid a repeated and/or pronounced deficit in the coverage of the daily calorie requirements, which, due to the frequently occurring yoyo effect, can have a known negative impact on weight management and also on people's physical and psychological state of people. Of particular importance in this case is the burning of muscle mass in order to obtain energy that usually takes place then, which in turn has a negative impact by reducing the basal metabolic rate and worsening physical fitness, and so forth.

Because a person employing the method is specifically urged to make sure to essentially cover his calorie requirements, the method according to the invention can also further distinguish itself from the prior art by making it possible to increase the motivation of the person to maintain a weight management program over a longer period of time. Lasting successes can thus be achieved, allowing the method to excel in comparison to various weight management guidelines, particularly in comparison to hypocaloric crash diets, naturally.

The method according to the invention also takes into account the fact that the calorie intake through meals and beverages in the course of a day does not in total exceed the daily caloric requirements of a person that corresponds to his individual life circumstances. This is a fundamental requirement if the goal is to lose weight.

If the end of a meal is taken as the beginning of an abstinence period only in the case of a meal after which the person has a subjectively sufficient feeling of satiety—i.e. in which a possibly present feeling of hunger has been satisfied—, then it is possible to ensure in a simple way that sufficient nutrition is being consumed and that the method according to the invention is being carried out under the desired conditions, thus also better taking into account individual requirements and habits of a person. Among other things, it does not matter how many meals a person eats until he has a corresponding feeling of satiety—it is thus also possible for an abstinence period to begin after the last of several small meals. This can likewise be used in the method with regard to maintaining muscle mass, fitness, etc. It is also possible to advantageously avoid development of a disturbed eating behavior—of the kind that can occur in the case of a desire to reduce weight.

If the time value is established as either a maximum of 6 hours during the wake phase of the person or a maximum of 12 hours during his sleep phase, then the method can more easily determine the time of a change in a metabolic state as a function of energy consumption. As explained above, there is a lower energy requirement in the sleep phase, which is why an insulin level is high for longer and the fat burning is reduced for a longer time. This circumstance is taken into account according to the invention. It is also possible to determine the time value individually—for example to reduce it in people whose bodies have an elevated basal metabolic rate or who exert themselves more in their work. It has turned out, however, that the above-mentioned change in metabolism can occur in practically everyone after the maximum values indicated.

In the method according to the invention, if physical activity is performed during the abstinence period, it is also possible to set the duration of the time value to end with the occurrence of a general fatigue and exhaustion of large parts of the skeletal muscles. This is particularly true in endurance- and strength/endurance sports—i.e. with athletic activity such as jogging, cycling, etc.—, but also in professional activity with corresponding intensity. The expression “large parts of the skeletal muscles” is understood to mean primarily the muscle groups that are responsible for locomotion and for holding the body upright. In this connection, the corresponding duration can specifically be assumed to be the amount of time that passes until the glycogen stores both in the involved muscles and in the liver are essentially depleted, the blood sugar level and subsequently also the insulin level have dropped, and consequently, a predominantly fat-burning metabolism, i.e. an active lipolysis as well, is occurring. As evidence for the end of this period, it is thus possible to use the time at which a restriction or reduction of the fitness occurs and/or it is necessary to take a break.

If up to one hour per day is added to the calculated results whenever the person engages in athletic activity for at least 20 consecutive minutes on that day, then it is possible to selectively take into account any life habits that are not included in the time values. In other words, durations of physical activity are detected, added up, and taken into account in the method, it being immaterial when the athletic activity takes place. The athletic activity can also be advantageously taken into account in a way that is comparatively independent of the type of activity—whether it be endurance training, strength/endurance training, or also strength training—so long as it is not of such a high intensity that it results in muscular fatigue or muscular exhaustion and thus requires taking a break. It is thus possible to individually take into account when the glycogen level of the body is depleted more quickly and an active lipolysis occurs at a correspondingly earlier point.

If the weight management takes into account a summation of only the calculated results with positive values, then abstinence periods with predominantly fat-burning metabolism are not reduced, even if one or more abstinence periods turn(s) out to be shorter than the corresponding time values. As a further consequence, days in which the weight management is not occurring in the desired way also do not result in a reduction of already added periods in which a reduction of the body's fatty tissues has already occurred. It is therefore also possible to improve the motivation of a person to carry out the method since positive results that have been achieved are retained.

If the weight management takes into account the value of the daily summation up to a maximum of 4 hours per day and a maximum of 15 hours per week, then it is possible to achieve improvements with regard to a person's motivation to carry out the method. It is thus possible to prevent a person from trying to compensate for the days on which the life habits do not occur in the way that is desired in the method by adding days that have a disadvantageous, excessively high number of hours in the summation. In addition, since a person knows that no additional time is added beyond these maximum values, this also makes it possible to control the motivation to improve life habits through realistically achievable goals in the case of a desire to reduce body weight. It is thus possible to better avoid the danger of a negative behavior that may possibly even be hazardous to health—possibly due to undesirably long abstinence periods.

If the consumption of foods and/or beverages that contain chemical energy resumes after the end of an abstinence period, which is calculated from the sum of the time value of this abstinence period plus 4 hours, then it is possible to better insure avoiding the danger of slipping into a metabolic state in which increased musculature reduction occurs. As explained above, the invention thus makes it possible among other things to achieve the fact that the basal metabolic rate of a person can remain high, physical fitness can be maintained, and also a muscle building that depends on increased athletic activity, for example, is not inhibited.

If breaks are provided in the detection of abstinence periods, then among other things, this makes it possible to enable moderate changes in a person's life- and/or dietary habits. It has turned out that the method achieves significantly longer-lasting successes than would be achieved with radical changes in a person's daily routine. In this connection, the method according to the invention can excel in comparison to the prior art also in that it provides steps that can be used to avoid psychological stresses or even compulsive behavior that can in the end even lead to a disturbed eating behavior, a reduction in social contacts, or the like. A break of 2 days after 5 consecutive days of nutritional abstinence detection has proven to be particularly advantageous.

If the weight management is controlled in accordance with detected parameters of the physical and/or psychological state of the person, it is then possible, as explained above, to address different conditions with regard to life- and nutritional situations, physical and mental fitness, etc. Likewise, the execution of the method can also take into account different goals to be achieved—for example with regard to the burning or maintaining of body fat. For example, time values can be established in accordance with these goals. It is also possible to adapt the method in simple, but effective ways to the results of the weight management or changes in accordance with the physical activity in the daily routine of a person. In the final analysis, the method according to the invention can also be used to better motivate a person by taking into account the person's well-being, which once again makes it possible to ensure a long-lasting success of the weight management.

The invention also includes an electronic device for carrying out the method, with an input device equipped with a display device, a time measuring device, a processing unit, and a memory connected to the processing unit. In this connection, it is possible in particular to provide a mobile terminal, which is embodied so that a person can wear it on his body, preferably like a wristwatch, so as to be able to simply carry out the method throughout the entire day. This is also advantageous in that in the course of a day, a person is informed of the status—for example the duration of a current abstinence period—or can possibly also receive positive motivation by means of the electronic device. It is thus possible for a person to carry out the method independently. This can likewise contribute to strengthening the personal responsibility of a person with regard to the weight management. In addition, a memory makes it possible for data to be saved and evaluated over a longer period of time, thus permitting the method to be correspondingly adapted to the data.

### DETAILED DESCRIPTION OF THE PREFERRED EMBODIMENTS

The method according to the invention will be explained in greater detail by way of example on the basis of embodiment variants.

In general, it can be stated that only in a metabolic state with activated lipolysis in the adipocytes of the body's fatty tissue is the requirement for reducing the mass of the body's fatty tissue met since the adipocytes of the fatty tissue are then putting out more energy to the blood to supply the body with energy than they are taking in from the blood—and their mass is reduced as a result.

A time measuring device is used to detect time intervals, which are incorporated into the weight management. In so doing, an abstinence period, between the consecutive times a person consumes food and/or beverages containing chemical energy is measured repeatedly. In other words, during an abstinence period, the body does not take in any calories in the form of foods or beverages. The drinking of water or, for example, unsweetened tea—in accordance with the individual feeling of thirst—can, however, take place at any time during the abstinence period, without causing it to end, since they do not contain any calories.

A time value, which depends on the lipid metabolism—in particular lipolysis in the fatty tissue—that is present during the abstinence period, is subtracted from the respective measured abstinence periods. In addition, the time value depends on the physical activity or on the sleep or wake state during the abstinence period. It is determined independently, however, of the person's respectively current calorie count in the course of a day. The results calculated from this are incorporated into the weight management. To that end, the person uses a mobile terminal with an input device, a display device, a time measuring device, a processing unit, and a memory connected to the processing unit.

The method will be explained below by way of example primarily in connection with a desired reduction in body fat. For the sake of completeness, it should be noted that in the case of a desired increase in body weight, the method according to the invention can be used to specifically avoid periods with activated lipolysis in the adipocytes of the fatty tissue.

### Example 1

A person ends a meal at 7:00 AM after which this person has a subjectively sufficient feeling of satiety, i.e. a previously existing feeling of hunger has been satisfied. The latter is a requirement for the beginning of an abstinence period whose time is detected by means of the input device, in this connection, it does not matter how many—for example smaller—meals it has taken to achieve this meal that produces a subjectively sufficient feeling of satiety at the beginning of a period of abstinence. Over a period of 8 hours, the body is then furnished with no energy in the form of calorie-containing foods or beverages and does not perform any medium-heavy work or heavy work—and also does not sleep.

After this, the person uses the input device to enter 3:00 PM as the end of the abstinence period since he consumes a chemical energy-containing meal again at 3:00 PM.

In this example, the period of 2 hours with activated lipolysis in the adipocytes of the fatty tissue from 1:00 PM to 3:00 PM during which a reduction in the mass of the body's fatty tissue occurs is automatically determined by the processing unit of the mobile terminal in that it subtracts the time value of 6 hours from the total abstinence period of 8 hours, calculates a summation of the time remaining after the expiration of the time value until the end of the abstinence period, and stores the summation for purposes of controlling the weight management.

In the person's wake phase, the time value is set as a maximum of 6 hours. The person can use an input device to detect the level of his physical activity and can thus actuate the input device in order to match the time value in the wake phase to the intensity of the physical exertion. Since the person in this example is not exerting or performing physically strenuous activity or work, the time value is set to the maximum value of 6 hours in accordance with the above description. In a variant of the method, it is possible to reduce the time value in the wake phase to 5 hours with predominantly medium-heavy work during the abstinence period and to reduce it to 4 hours with predominantly heavy work during the abstinence period.

### Example 2

A person who does not exert or perform any physical activity or work ends a meal at 6:00 PM after which this person has a subjectively sufficient feeling of satiety. The person uses the input device to define this time as the beginning of the abstinence period. Over a period of 14 hours, the body does not ingest any energy in the form of calorie-containing foods or beverages. In this case, the person uses the input device to define 8:00 AM the next day as the end of the abstinence period since another meal will be eaten at 8:00 AM, and with the input device, confirms that the sleep phase took place during the abstinence period between 6:00 PM the day before and 8:00 AM the next day.

In this example, the period of 2 hours with activated lipolysis in the adipocytes of the fatty tissue from 6:00 AM to 8:00 AM during which a reduction in the mass of the body's fatty tissue occurs is automatically determined by the processing unit of the mobile terminal, in that it subtracts the time value of 12 hours from the total abstinence period of 14 hours, calculates a summation of the time remaining after the expiration of the time value until the end of the abstinence period, and stores the summation for purposes of controlling the weight management.

During the sleep phase, the body's glycogen level is depleted more slowly due to the lower energy consumption in comparison to a wake phase. This circumstance is taken into account by the longer time value in the sleep phase in that it is set to up to a maximum of 12 hours in the sleep phase.

The periods of 2 hours (from 1:00 PM to 3:00 PM) in example 1 and 2 hours (from 6:00 AM to 8:00 am) in example 2 are incorporated into the weight management. They are determined by the processing unit of the mobile terminal and are recorded in a memory connected to the processing unit. In addition, a summation of only those calculated results with positive values takes place. In other words, this constitutes a summation of the periods in which activated lipolysis in the adipocytes of the fatty tissue results in a reduction in the mass of the body's fatty tissue. This has a significantly positive influence on the motivation of the person to carry out the method for a longer period of time.

In the case of a desired reduction in body weight, it is recommended to achieve a total of 10 to 15 hours per week in which activated lipolysis occurs in the adipocytes of the fatty tissue. In order for the method not to cause radical changes in life- and dietary habits, the daily summation of up to a maximum of 4 hours per day and a maximum of 15 hours per week is taken into account. The person is also informed of these maximum values; as a result, experience has shown that the method is also better able to achieve longer-lasting successes.

### Example 3

At 8:00 AM, a person ends a meal that contains chemical energy and uses the input device to record this time as the beginning of an abstinence period. At 9:00 AM, this person begins endurance training in the form of jogging, at a stress level that is at the boundary between the aerobic and anaerobic range. After 40 minutes of exercise, fatigue of large parts of the skeletal muscles, in particular those of the legs, sets in so that the person takes a break. It can therefore be assumed that this person has essentially depleted his glycogen level so that the body has switched to a predominantly fat-burning lipid metabolism in order to produce energy. According to the invention, this results in the establishment of a time value of 1 hour 40 minutes. Since the abstinence period ends at 12:40 PM. according to the invention, a period of 3 hours is incorporated into the weight management.

In addition, however, physical activity that has not resulted in muscular fatigue can also be incorporated into the method. To that end, a maximum of one hour per day can be added to the calculated results if on this day, the person engages in athletic activity, particularly in the form of endurance-, strength/endurance- and/or strength training, for a period of at least 20 consecutive minutes. For example, the person from example 3, on the day after the above-described endurance training, did two muscle-building workouts of 40 minutes each. For this, his additional 60 minutes are added to the hours calculated the previous day for the weight management—this number of minutes, however, can depend on factors such as the type, intensity, and/or duration of the workout as well as individual factors such as weight, gender, etc. This also contributes, however, to motivating the person to engage in athletic activities.

If the consumption of foods and/or beverages that contain chemical energy is set to start after an abstinence period that is composed of the sum of the time value of this abstinence period and 4 hours, then it is possible to prevent the method from triggering disadvantageous—primarily protein-burning—metabolic states in the person. Likewise, in order to avoid undesirable effects of an excessively radical change in the life- and dietary habits due to excessively long periods with active lipolysis in the adipocytes of the fatty tissue, etc., breaks are provided in the abstinence period detection, in particular for 2 days after 5 consecutive days of nutrition abstinence detection. The person is informed of this.

If the weight management is controlled in accordance with detected parameters of the physical and/or psychological state of the person—it is possible, for example, to imagine an option for inputting these states—, then the method can be advantageously adapted to the respective development. It can thus be useful, among other things, to provide longer breaks, for example if the person has motivation problems.

Consequently, the method according to the invention makes it feasibly possible for a person who wishes to lose weight to be provided with information about how long the requirements for reducing the body's fatty tissue have been met. The person is therefore also provided with control over how often the requirements for reducing the body's fatty tissue have to be planned and carried out in order to reach the recommended 10-15 hours per week in which activated lipolysis in the adipocytes of the fatty tissue results in a reduction in the mass of the body's fatty tissue.

1. A method for personal weight management, comprising:
using a time measuring device to measure abstinence periods between consecutive times that a person consumes food and/or beverages containing chemical energy and incorporating the times into the weight management; and
subtracting a time value, which depends on a lipolysis of lipid metabolism—that is present during an abstinence period, from the respective measured abstinence periods, and incorporating results calculated from this subtraction into the weight management.

2. The method according to claim 1, wherein the time value is defined as a period that lasts from a beginning of an abstinence period until a person's metabolism switches to a predominantly fat-burning state.

3. The method according to claim 1, wherein the time value depends on a physical activity or on a sleep or wake state during the abstinence period.

4. The method according to claim 1, wherein the time value for the person is determined independently of his respectively current calorie count in the course of a day.

5. The method according to claim 1, wherein an end of meals is taken as a beginning of an abstinence period only if, in total, the meals in a day essentially cover the daily calorie requirements corresponding to the life circumstances of the person and do not go beyond these calorie requirements.

6. The method according to claim 1, wherein an end of a meal is taken as a beginning of an abstinence period only in a case of a meal after which the person has a subjectively sufficient feeling of satiety.

7. The method according to claim 1, wherein the time value is established as either a maximum of 6 hours during a wake phase of the person or a maximum of 12 hours during his sleep phase.

8. The method according to claim 1, wherein if endurance- or strength/endurance-type physical activity is performed during the abstinence period, a duration of the time value ends with an occurrence of a general fatigue and exhaustion of large parts of the person's skeletal muscles.

9. The method according to claim 1, further comprising adding up to one hour per day to the calculated results whenever the person engages in any endurance-, strength/endurance, or strength training athletic activity for at least 20 consecutive minutes on that day.

10. The method according to claim 1, wherein the weight management takes into account a summation of only the calculated results with positive values.

11. The method according to claim 1, wherein the weight management takes into account a value of a daily summation up to a maximum of 4 hours per day and a maximum of 15 hours per week.

12. The method according to claim 1, comprising resuming a consumption of foods and/or beverages that contain chemical energy after the end of an abstinence period, which is calculated from the sum of the time value of this abstinence period plus 4 hours.

13. The method according to claim 1, comprising providing breaks in the abstinence period detection, for 2 days after 5 consecutive days of nutrition abstinence detection.

14. The method according to claim 1, comprising controlling the weight management in accordance with detected parameters of the physical and/or psychological state of the person.

15. An electronic device for executing the method according to claim 1, the electronic device comprising a mobile terminal having an input device, a display device, a time measuring device, a processing unit, and a memory connected to the processing unit.
